(19) United States
(12) Patent Application Publication
(10) Pub. No.: US 2004/0037904 A1
(43) Pub. Date: Feb. 26, 2004

(54) Antibacterial composition comprising as an active ingredient saponificated vegetable oil

(76) Inventor: Lennart Hakansson, Taby (SE)

Correspondence Address: DINSMORE & SHOHL, LLP, 1900 CHEMED CENTER, 255 EAST FIFTH STREET, CINCINNATI, OH, 45202, US

(21) Appl. No.: 10/333,997

(22) PCT Filed: Jul. 26, 2001
(86) PCT No.: PCT/SE01/01676

(30) Foreign Application Priority Data
Jul. 27, 2000 (SE) 0002779-7
Feb. 1, 2001 (SE) 0100299-7

(51) Int. Cl. A61K 35/78; A61K 31/202

(52) U.S. Cl. 424/757; 514/560; 424/764; 424/755

(57) ABSTRACT

The present invention relates to an antibacterial composition, comprising as an active ingredient saponificated vegetable oil, preferably soybean oil, or one or more components thereof; and at least one carrier. Also a ready-to-use composition comprising a with water diluted composition and a process for the production of an above composition is disclosed. Use of a saponificated vegetable oil, preferably soybean oil, or one or more components thereof is disclosed. Additionally the present invention relates to a method for controlling bacteria on pests comprising the application of an anti-bacterially effective amount of an above composition onto said pests.

The present invention relates to an antibacterial composition, comprising as an active ingredient saponificated vegetable oil, preferably a soybean oil, or one or more components thereof; and at least one carrier. Also a ready-to-use composition comprising a with water diluted above composition and a process for the production of an above composition is disclosed. Use of a saponificated vegetable oil, preferably soybean oil, or one or more components thereof is disclosed. Additionally the present invention relates to a method for controlling bacteria on pests comprising the application of an anti-bacterially effective amount of an above composition onto said pests.

### BACKGROUND TO THE INVENTION

There are certain compositions previously known for controlling pests on animals, e.g. through EP 794701 where the control of pests using pyretroid compositions is disclosed. However, nothing is disclosed regarding using saponificated soybean oil, or saponificated vegetable oil, for anti-bacterial treatment of bacteria on pests. A drawback with this is the complex nature of the composition requiring an emulsifier.

Soy hydrolysate has been disclosed for use in insect control of trees in WO99/63840. Phosphatides from soybean oil have been disclosed for use in controlling grain insects (U.S. Pat. No. 4,721,706). However, neither of these documents is disclosing the use of saponificated soybean oil, or saponificated vegetable oil, for anti-bacterial treatment of bacteria on pests. A disadvantage with the compositions in WO99/63840 is the necessity of using an enzyme for hydrolysing soy. The soybean phosphatides used in U.S. Pat. No. 4,721,706 is separated with several process step, thus a complex refining process is necessary, which obviously is a drawback.

Further, an arthropod control composition for plant protection is disclosed through EP 583774. The composition may comprise soybean oil, but this is complex through the use of several components. However, nothing is disclosed regarding using saponificated soybean oil, or saponificated vegetable oil, for anti-bacterial treatment of bacteria on pests.

Additionally, acaricide compositions are disclosed in JP56008308 where soybean oil with a saponification equivalent of 176-286 and iodine value of 11-214 may be used together with an emulsifier for killing insects on fruits. However, nothing is disclosed regarding using saponificated soybean oil, or saponificated vegetable oil, for anti-bacterial treatment of bacteria on pests. A drawback is here the necessity of an emulsifier.

Thus there is in view of the above mentioned documents a need for new compositions for the control of bacteria on pests, which lack the drawbacks mentioned above.

### SUMMARY OF THE INVENTION

The present invention solves the above problems by providing an antibacterial composition, comprising as an active ingredient saponificated vegetable oil, preferably soybean oil, or one or more components thereof; and at least one carrier. Also a ready-to-use composition comprising a with water diluted composition and a process for the production of an above composition comprising saponification of a vegetable oil, preferably soybean oil, by adding an alkali hydroxide thereto, preferably potassium hydroxide; and adding a carrier is disclosed. Use of a saponificated vegetable oil, preferably soybean oil, or one or more components thereof in medicine and use of a saponificated vegetable oil, preferably soybean oil, or one or more components thereof in the manufacture of an agent for treating bacteria is also disclosed. Additionally a method for controlling bacteria on pests comprising the application of an anti-bacterially-effective amount of an above composition onto said pests.

### DETAILED DESCRIPTION OF INVENTION

With the expression “vegetable oil” is meant in the present description an oil selected from the group consisting of soybean oil, rape seed oil and sunflower oil or a mixture thereof. These three vegetable oils have similar fatty acid patterns (see “Matfettslexikon-fr\dot{a}n frö till bytta” ed. H. Korp, N Lindskoug, Bokhuset och Margarinfo, 1984, pp. 80, 88 and 89; hereby incorporated by reference thereto). Preferably soybean oil is used in the composition according to the present invention.

With the expression “saponificated” is meant in the present description that the vegetable oil, preferably soy bean oil, has been converted, preferably by using an alkali hydroxide, from a vegetable oil to a soap. The alkali hydroxide may be sodium hydroxide or potassium hydroxide, preferably potassium hydroxide, thus rendering into potassium oleate. Preferably 98% of the fatty acid(s) in the composition is (are) saponificated.

With the expression “carrier” is meant in the present description an organic or inorganic carrier which may be natural or synthetic, and which is associated with the active ingredient and which preferably facilitates its application to the pests to be treated. This carrier is thus generally inert and may preferably be a veterinarily or pharmaceutically acceptable carrier. The carrier may be water, alcohols, ketones, polar solvents, aromatic solvents, oil solvents, saturated or unsaturated hydrocarbons, chlorinated hydrocarbons, liquefied petroleum gas or mixtures thereof; preferably water is the carrier. Pharmaceutically acceptable carriers are well known in the art. Exemplary of liquid pharmaceutically acceptable carriers are sterile aqueous solutions that contain no materials in addition to the active ingredients and water, or contain a buffer such as sodium phosphate at physiological pH value, physiological saline or both, such as phosphate-buffered saline.

According to one preferred embodiment of the present invention the composition comprises a potassium oleate, and the composition may preferably have an altered fatty acid pattern rendering into an enhanced anti-microbial effect. This altered pattern may preferably be formed by an increased amount of at least one fatty acid with a short carbon chain, which may preferably be at least one fatty acid with a carbon chain from C₁₂ (lauric acid) to C₁₈ (stearic acid), most preferred C₁₄ (myristic acid), thus rendering into an enhanced anti-microbial effect.

According to one preferred embodiment of the present invention the composition has an amount of at least one fatty acid with a short carbon chain from approximately 0.01 to 50% (weight), preferably from 0.1 to 5% (weight), most preferred from 2 to 4% (weight). All percentage amounts, regarding the composition according to the present invention, appearing in the present description are percentages by weight (if not other is stated).

According to one preferred embodiment of the present invention the composition has an amount of approximately 2% (weight) C₈ (caprylic acid), 2% (weight) C₁₀ (capric acid), 2% (weight) C₁₂ (lauric acid) and/or 2% (weight) C₁₄ (myristic acid).

According to one preferred embodiment of the present invention the composition has an amount of approximately 6% (weight) with fatty acids ranging (when regarding the number of carbons) from C₆ (caproic acid) to C₁₄ (myristic acid).

According to yet another preferred embodiment of the present invention, the composition according to the present invention contains an increased amount of at least two fatty acids with a short carbon chain, preferably one fatty acid with a carbon chain of C₁₂ (lauric acid) and one fatty acid with a carbon chain with a length from C₁₄ (myristic acid) to C₁₈ (stearic acid), wherein the amounts for each fatty acid may be as in the above earlier mentioned preferred embodiment.

The composition according to the present invention may comprise:

a) from 0.1 to 50% of saponificated vegetable oil, preferably potassium oleate,

b) optionally one or more other fatty acid(s) which may be saponificated, and

c) water for the rest, totalling 100%;

preferably 25% potassium oleate and 75% water.

The composition according to the present invention may also be pesticidal and/or anti-ovulating.

According to yet another preferred embodiment of the present invention there is provided a ready-to-use composition comprising a with water diluted composition according to the present invention, wherein the composition according to the present invention may be diluted with water giving a relationship composition: water from 1:20 to 1:40. For application to pests preferably a diluted composition according to the present invention is used. Compositions “concentrated” according to the invention may preferably be commercialised, transported or stored.

According to yet another preferred embodiment of the present invention there is provided a process for the production of a composition according to the present invention comprising saponification of a vegetable oil, preferably soybean oil, by adding an alkali hydroxide thereto, preferably potassium hydroxide; and adding a carrier. The fatty acids are extracted through a basic hydrolysis of the oils and these fatty acids regulates the length of the carbon chains. In the saponification process the hydrogen in the COOH-group is split and replaced by a potassium-ion. The process has a stable equilibrium that demands a surplus of OH-ions, which means that a soap or a soft soap solution generally is basic.

According to yet another preferred embodiment of the present invention there is provided a saponificated vegetable oil, preferably soybean oil, or one or more components thereof for medical use.

According to yet another preferred embodiment of the present invention there is provided use of a saponificated vegetable oil, preferably soybean oil, or one or more components thereof in the manufacture of an agent (a medicament) for treatment, prevention or management of bacterial, viral or fungal diseases, preferably bacterial.

According to yet another preferred embodiment of the present invention there is provided use of a saponificated vegetable oil, preferably soybean oil, or one or more components thereof for inhibiting ovulation of pests or for repelling of pests.

According to yet another preferred embodiment of the present invention there is provided a method for controlling bacteria on pests comprising the application of an anti-bacterially effective amount of a composition according to the present invention onto said pests. The pests may be scabies, insects, arachnids, arthropod, nematode, helminth and protozoan pests, thrips, mites, weevils, aphids, pear suckers, ants, termites, flies, ticks, mealy bugs, dandruff, pollen (blossom) beetle (Meligethes aeneus), lice or fleas (or fungi as set out below), preferably scabies, flies (e.g. Musca domestica), lice, pollen (blossom) beetle, ticks or fleas. The pests may be infesting or suspected to infest an animal or a human being, whereby the composition according to the present invention may be termed a dermatology composition. Preferably the animal is selected from the group comprising dogs, cats, horses, camels and other pets with furs or poultry (which may be carriers of mites, which in turn may be carriers of Salmonella bacteria). The pests may also be infesting or suspected to infest a locus preferably a barn or a stable. The pests may be exposed to the composition according to the present invention when in nymph, larvae or fully grown-up phases.

According to yet another preferred embodiment of the present invention there is provided a method for controlling bacteria on pests comprising the application of an anti-bacterially effective amount of a composition according to the present invention onto said pests, wherein the pests may be a fungus, e.g. scurf, powdery mildew (Sphaerotheca humuli) or downy mildew (Pseudoperonospora humuli). The fungi may e.g. be carriers of Salmonella bacteria. The fungi may be infesting or suspected to infest animals or loci as set out above or fields for cultivation of e.g. crops. The fungi may further be infesting or suspected to infest crops as such, which may be standing crops or crop. The crops may e.g. be potatoes, rape seed, species belonging to the family Brassica, mustard, Swedish turnip, rutabaga, cabbage (red or white), Brussels sprouts or hops.

The composition according to the present invention may preferably be applied to the pests through the use of spraying. Preferably a diluted composition, previously made from more concentrated formulations, is used for the spraying. Preferably a fine spray with small droplets is used, which may be obtained by using high pressure and a nozzle with appropriate size. The composition according to the present invention may further be present in a spray bottle.

The composition according to the present invention may further be used as an admixture of other active substances e.g. a pesticide, which may be an insecticide, acaricide, fungicide or herbicide.

A farther advantage with the composition according to the present invention is the non-necessity for using an additive which normally is the case when adapting a soft soap or soap solution for use as an insecticide in order to get a water/fat balance. Another advantage is that the composition according to the present application is environmentally friendly compared to other pesticides. The composition is easily degradable in the nature, inexpensive, non-toxic and not prone to the effects of genetic resistance.

We will now describe the present invention by using figures and examples but they are only for purposes of illustration and shall not in any way limit the scope of the appended set of claims.

### FIGURES

FIG. 1 shows the mortality percentage when using RB 86™ (azadiraktin, a herb extract), against Musca domestica (adult).

FIG. 2 shows the mortality percentage when using Bionim™ (azadiraktin) against Musca domestica (adult).

FIG. 3 shows the mortality percentage when using emulsifier against Musca domestica (adult).

FIG. 4 shows the mortality percentage when using potassium oleate against Musca domestica (adult).

FIG. 5 shows the mortality percentage when using Blidol™ (a washing agent), against Musca domestica (adult).

FIG. 6 shows the control with larvae in water for comparison with FIGS. 7-11.

FIG. 7 shows the mortality percentage when using RB 86™, against Musca domestica (larvae).

FIG. 8 shows the mortality percentage when using emulsifier, against Musca domestica (larvae).

FIG. 9 shows the mortality percentage when using Bionim™, against Musca domestica (larvae).

FIG. 10 shows the mortality percentage when using Blidol™, against Musca domestica (larvae).

FIG. 11 shows the mortality percentage when using oleate, against Musca domestica (larvae).

FIG. 12 shows spiracle opening for M. domestica with non-affected bristles.

FIG. 13 shows spiracle opening for M. domestica treated with Blidol™. The bristles are deformed.

FIG. 14 shows spiracle opening for M. domestica treated with Bionim™. The bristles are deformed.

FIG. 15 shows spiracle opening for M. domestica treated with oleate. The bristles are minimally affected.

### EXAMPLES

### Example 1

Control of Flies in Stables

The following example had the purpose of studying effects on flies using extracts of herbs and fatty acids. The tested substances were:

a) RB 86™ (azadiraktin, a herb extract from seeds of Azadirachta indica), 6% concentration

b) Bionim™ (azadiraktin), 0.4% concentration

c) Emulsifier, 6% concentration

d) Potassium oleate, 5% concentration

e) Blidol™ (a washing agent), 0.02% concentration

f) Control (water)

As RB 86™ needs an emulsifier for dissolving this emulsifier was also tested. All tests were reiterated, making a total of five times for each. Throughout the study, houseflies (Musca domestica) of a laboratory strain (772a) obtained from Statens Skadedyrslaboratorium, Lyngby, Denmark, was used. Standard medium for culturing larvae consisted of baker's yeast, mill: powder, water and wheat bran.

Adult Flies

Test vessels were Petri dishes made of glass with a diameter of 9 cm. With the help of a spraying bottle the bottoms of the vessels were treated with each of the compositions a)-f). After transferring 10 adult flies to each of the pre-treated dishes, the dishes were covered with plastic film and the registration started. Observations were performed by counting the surviving flies at the start, after 5 minutes and subsequently every 10^{th} minute up to a total of 60 minutes. Two further observations were made after 1.5 and 2 hours from the start, respectively.

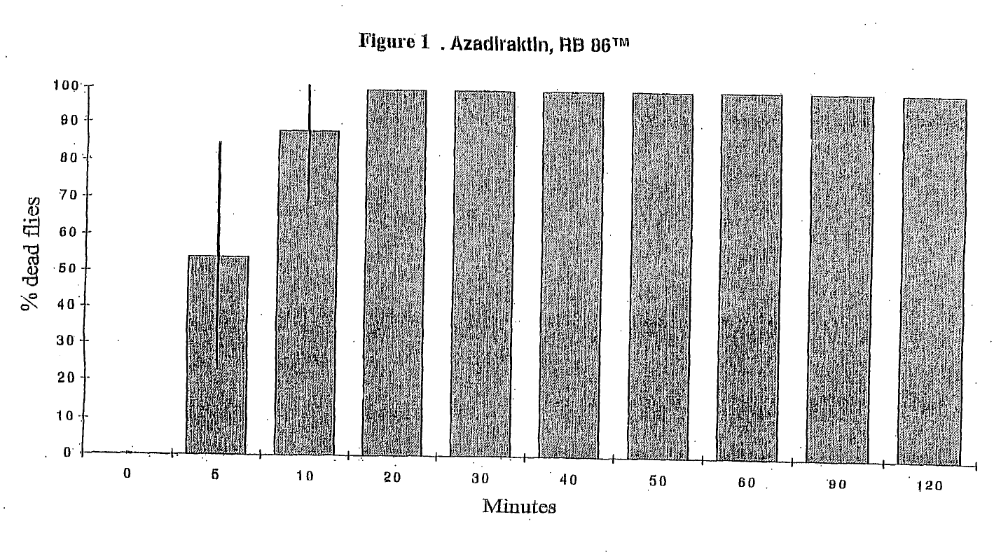

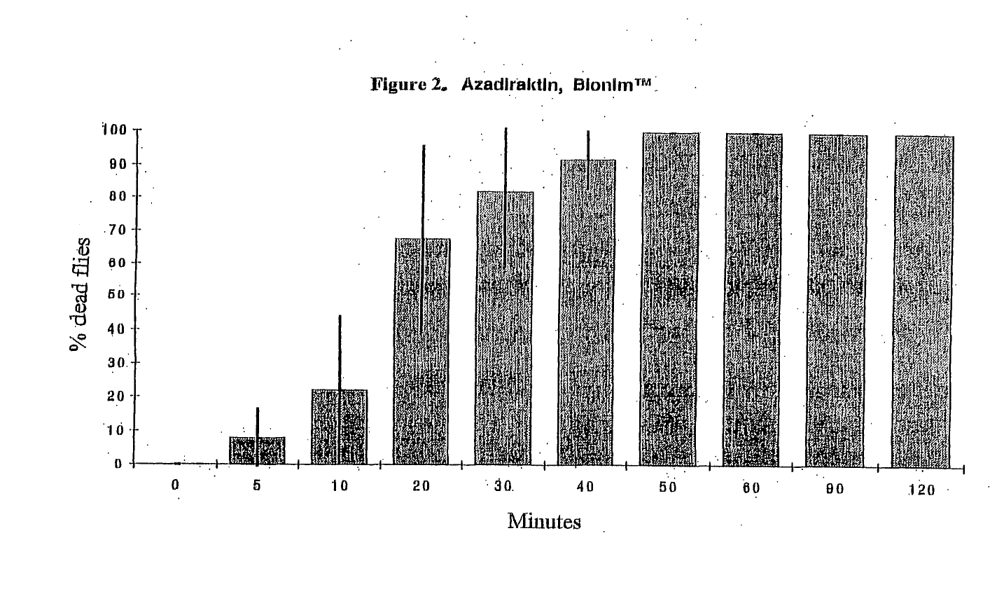

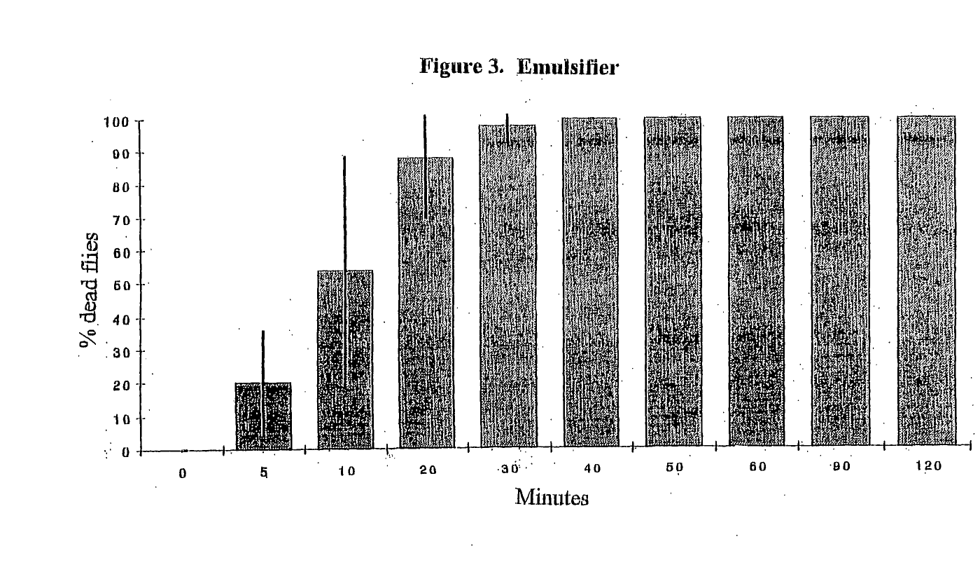

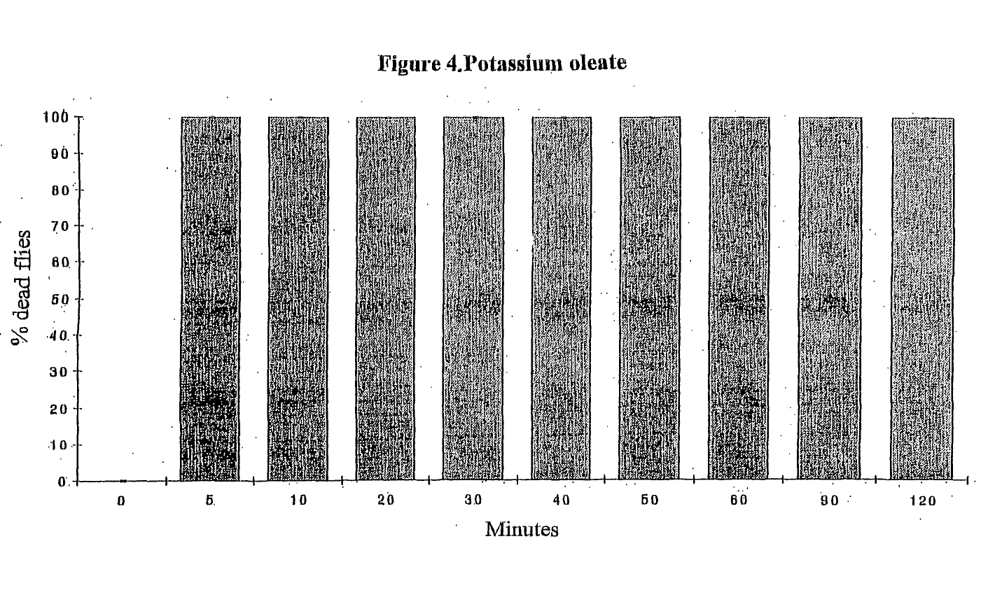

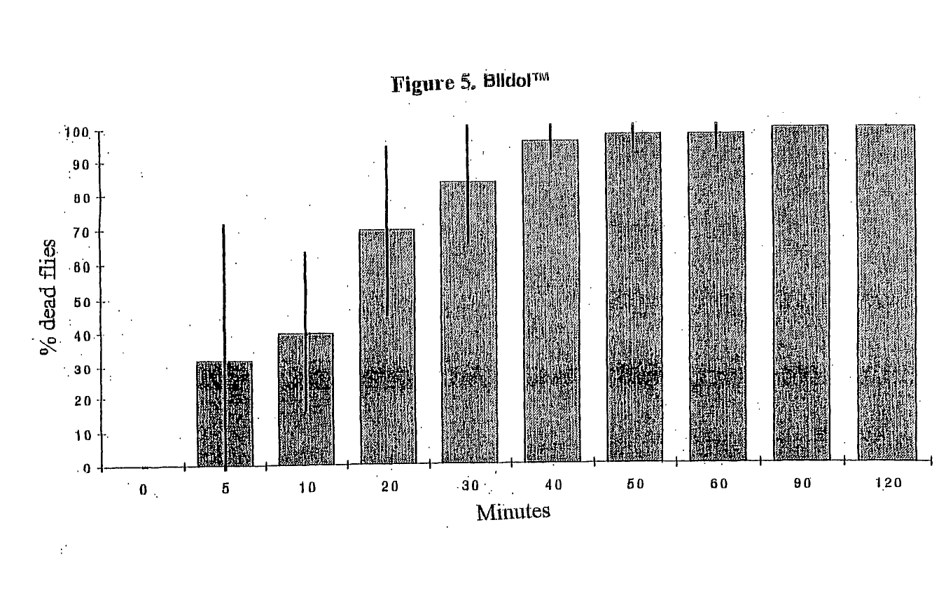

No flies within the control group died during the test. The both azadiraktin preparations had an effect whereby all flies were killed within 20 minutes (see FIG. 1) and 50 minutes (see FIG. 2), respectively. This supports one of Azadirachta indicas known characteristics namely being a negatively affecting agent for pests. Also an insecticide effect was detected for the emulsifier (see FIG. 3). Potassium oleate was the most efficient substance as all the flies were killed already within 5 minutes (see FIG. 4). Blidol™ had also a lethal effect (see FIG. 5).

Larvae

Six days old larvae were placed in 1.5 litre plastic vessels (Cerbo™) containing 40 g culture medium and the respective composition a)-f). The vessels were placed in a climate room (24° C., RH %=75%±10%). Observations were performed whereby the number surviving larvae were registrated after 2, 5, 12, 24 and 36 hours.

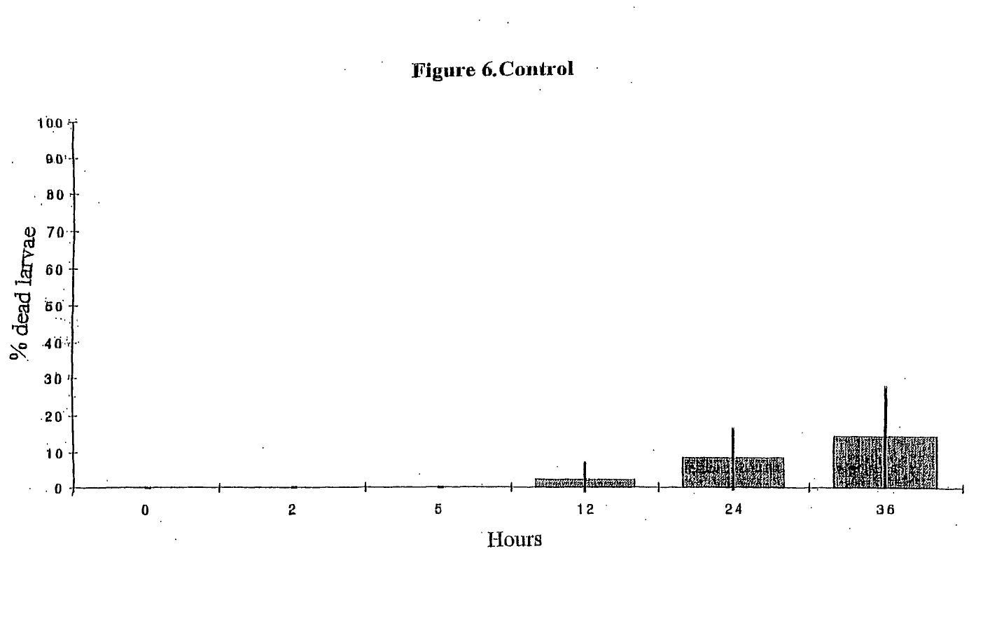

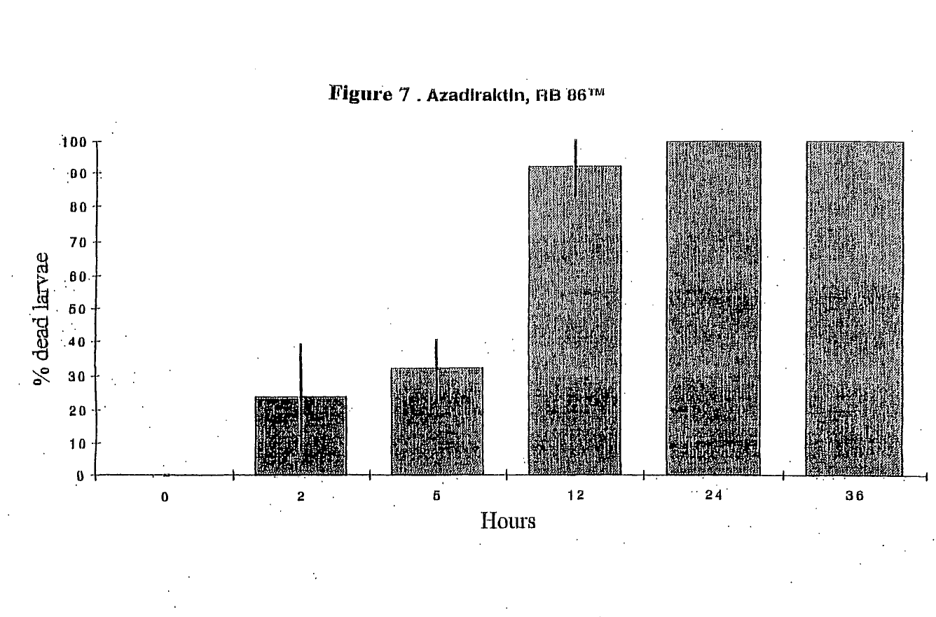

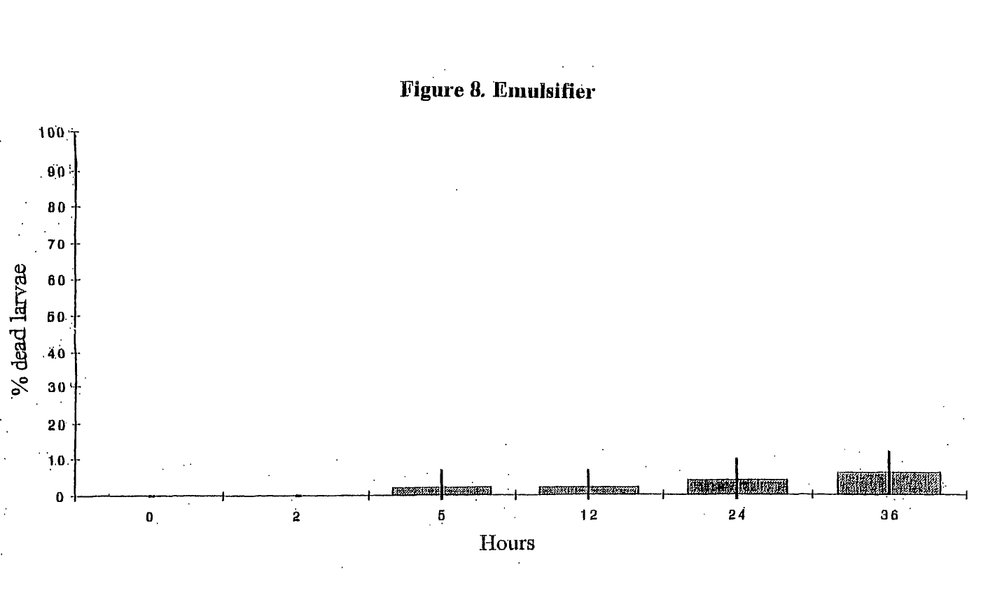

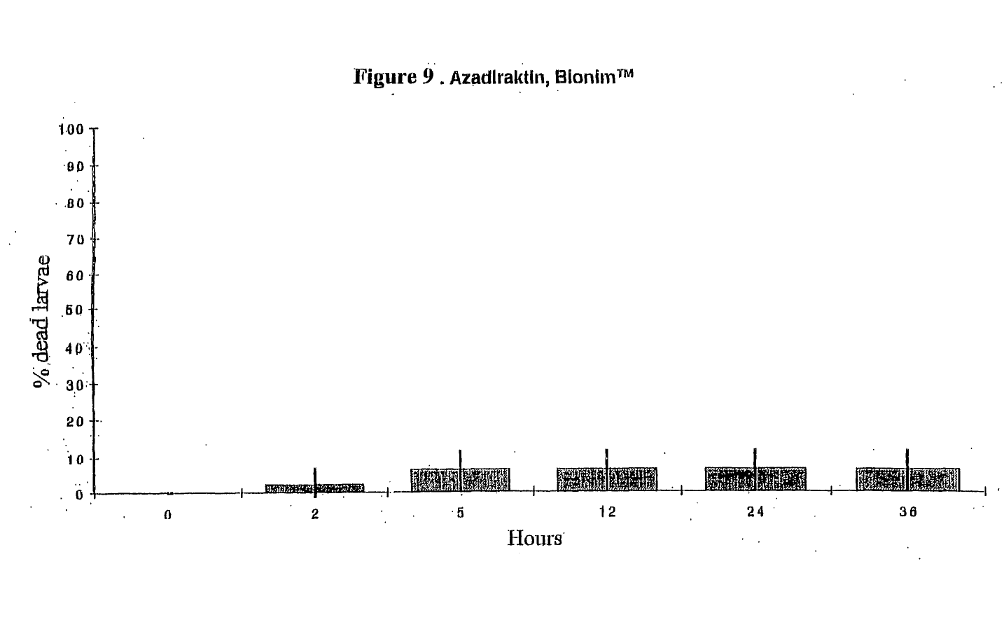

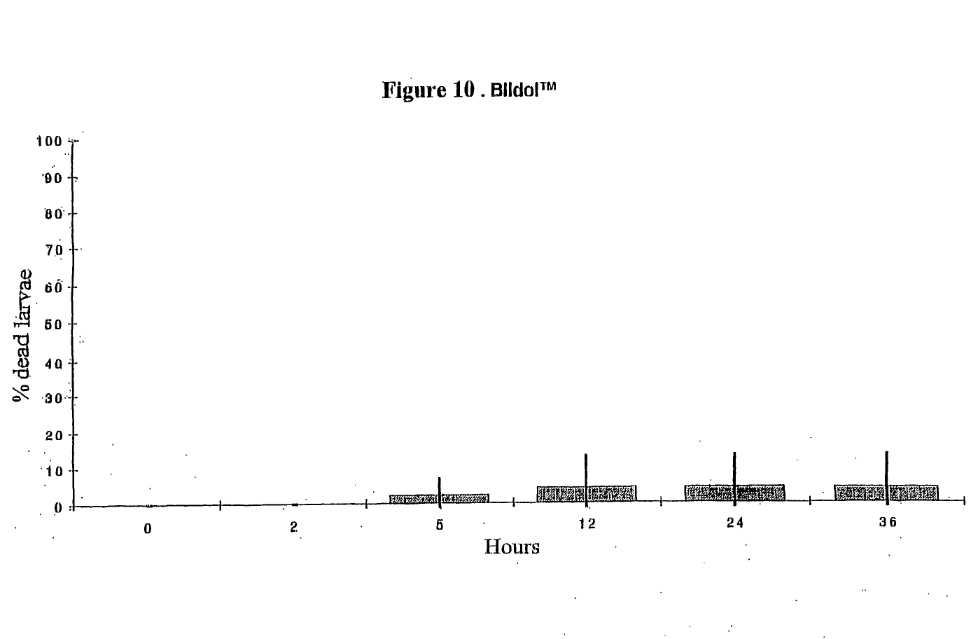

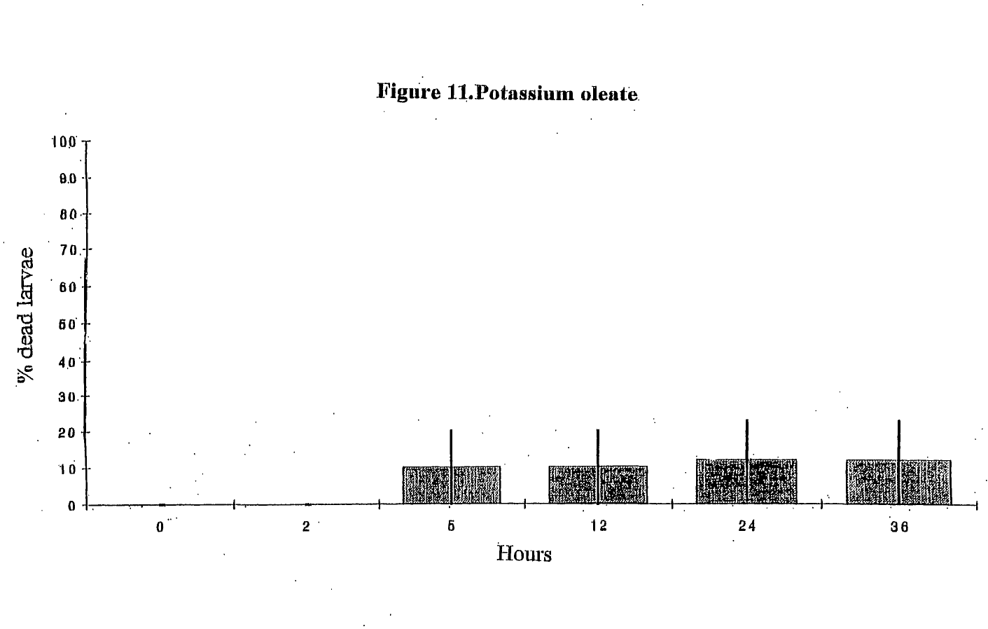

In the control group it was noted that 14% of the larvae died within 36 hours (see FIG. 6). The larvae were also sensible for Azadiraktin, RB86™ (see FIG. 7). Here the sticky consistency of the preparation may be a decisive factor. A controlling effect could not be detected for either of the compositions c) (see FIG. 8), b) (see FIG. 9) or e) (see FIG. 10). An effect could however be detected of the oleate (see FIG. 11).

### Example 2

Effects on Flies Studied in Scanning Electron Microscope

A scanning electron microscope (SEM) was used to microscope adult flies treated with:

i) Bionim™ (azadiraktin), 0.4% concentration

ii) potassium oleate, 5% concentration; and

iii) Blidol™ (a washing agent), 0.02% concentration,

whereby morphological changes could be detected. These flies were photographed and compared with normal flies.

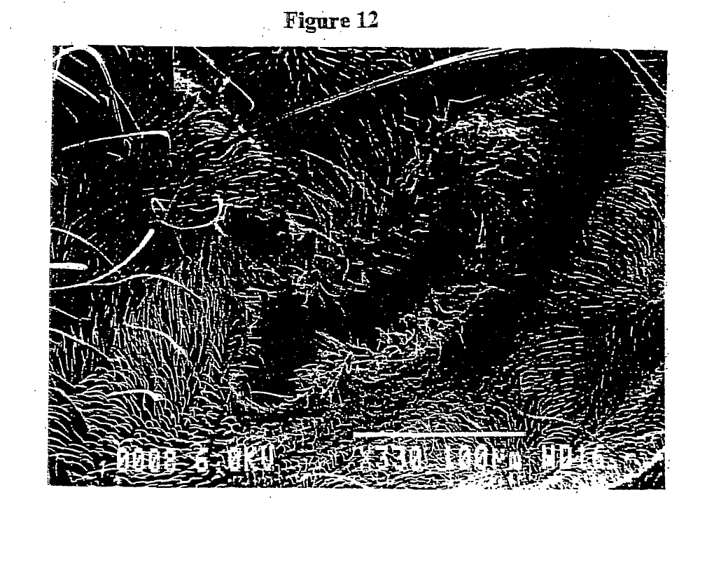

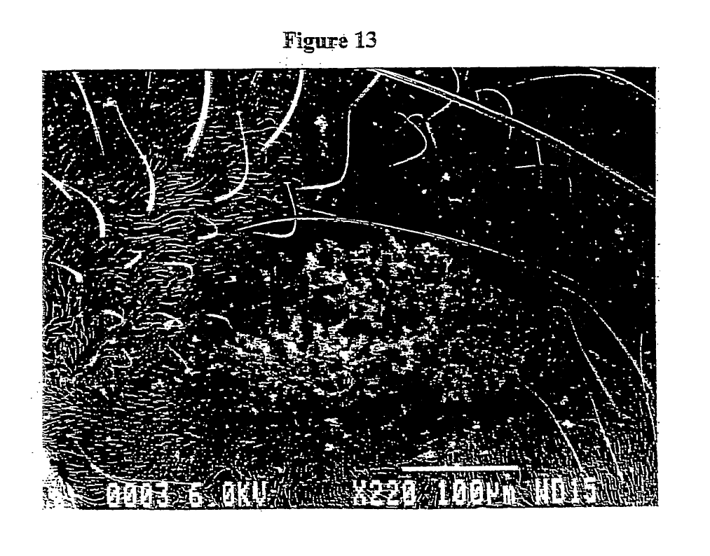

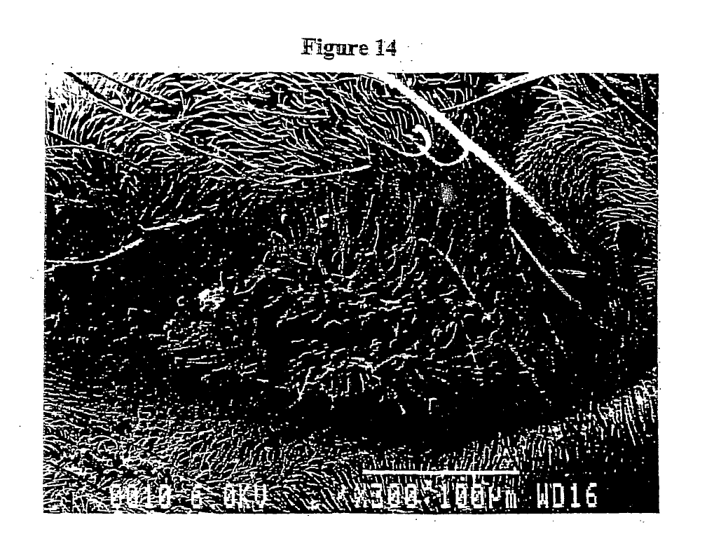

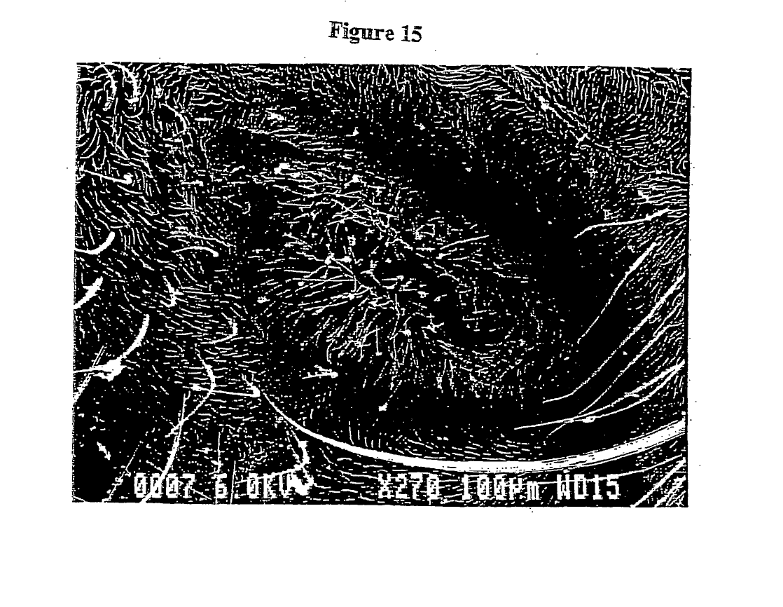

Morphological changes could be detected on the spiracle opening on the middle part of the body. Bristles (see FIG. 12) surround this breathing hole. Flies treated with the test compositions showed different levels of deformation on these bristles. Flies treated with Blidol™ showed a marked deformation on these bristles (see FIG. 13) while azadiraktin caused deformations in less extent (see FIG. 14). It was remarkable that flies treated with oleate only showed minimal changes in the bristles (see FIG. 15) as this substance is thought to solve chitin (in the exo-skeleton of insects), block spiracles and thus choke the insect. Apparently these last mentioned effects are present when using the other compositions.

### Example 3

Flea Control on Dogs

15 dogs infested with fleas were treated with oleate. The oleate was sprayed from pump bottles. At the first treatment the dogs were sprayed with oleate in the groins, armpits and head/neck. At the second treatment, twenty-four hours later, the dogs were sprayed only in the armpits and behind the ears.

The result was that after the first treatment only one or two fleas were found on some dogs (those badly infested with fleas). Forty-eight hours later, after the second treatment, no living flea was found. When another check was made four days later the result was the same.

### Example 4

Pollen Beetle Control on Rape Seed

In this experiment the effect of Bionim™ and Puricide (a composition according to the present invention), respectively was assessed on pollen beetles. Both compositions had an inhibiting effect on pollen beetles regarding their detrimental effect on rape buds and also on their eating capacity. The composition according to the present invention, however, had a stronger detrimental effect on the beetles in terms of their ovulation capacity, the number of eggs and their eating capacity. Thus a clear anti-ovulation (ovulation damaging) effect could be seen. The composition according to the present invention also had a stronger repelling effect.

The experiment essentially comprised two different methods:

1) ovulation and eating method

2) repelling method

In order to determine the effect of Bionim™ and Puricide on ovulation and eating, rape buds treated with Bionim™ and Puricide (and also control buds not treated with any of said compositions) were placed in cages. Pollen beetles were then guided into the cages. The buds were studied during two days under microscope, wherein differences could be assessed. The number of buds damaged through eating, the number of buds with ovulation damages and the number of eggs was assessed.

In order to determine the effect of Bionim™ and Puricide on keeping the buds free of beetles two small cultivation fields were used were white mustard was grown on one and rape seed on the other. The fields was divided into 24 quadratic sections wherein a third was treated with Bionim™, one third with Puricide and the last third was left untreated. The number of beetles was counted 12 hours and 24 hours after the commencement of the trial. Puricide had the strongest repelling effect, and this was especially marked after 12 hours.

The results of example 4 are given below as follows:

Results

The standard deviations are the values given in brackets.

Method 1—All Three Sets of Results Given Individually

TABLE 1

| | number of | | | | Number | |
|---|---|---|---|---|---|---|
| | ovulation | | Number | | of eating | |
| | damages/bud | | of eggs/bud | | damages/bud | |
| | (mean) | | (mean) | | (mean) | |
| 07 June | | | | | | |
| O | 0.13 | (0.128) | 0.21 | (0.256) | 0.15 | (0.091) |
| P | 0.04 | (0.067) | 0.10 | (0.224) | 0.02 | (0.026) |
| N | 0.06 | (0.061) | 0.15 | (0.208) | 0.11 | (0.072) |
| 19 July | | | | | | |
| O | 0.16 | (0.142) | 0.56 | (0.672) | 0.17 | (0.136) |
| P | 0.08 | (0.065) | 0.19 | (0.152) | 0.04 | (0.030) |
| N | 0.17 | (0.132) | 0.58 | (0.576) | 0.10 | (0.084) |
| 21 June | | | | | | |
| O | 0.10 | (0.129) | 0.21 | (0.433) | 0.28 | (0.183) |
| P | 0.02 | (0.025) | 0.01 | (0.022) | 0.09 | (0.071) |
| N | 0.02 | (0.047) | 0.03 | (0.092) | 0.13 | (0.126) |
| 26 June | | | | | | |
| O | 0.04 | (0.058) | 0.03 | (0.054) | 0.21 | (0.100) |
| P | 0.00 | (0.000) | 0.00 | (0.000) | 0.12 | (0.137) |
| N | 0.03 | (0.041) | 0.03 | (0.056) | 0.11 | (0.067) |

O = untreated
P = treated with Puricide
N = treated with Bionim ™

Method 1—Mean Values from the Three Sets

TABLE 2

| | number of | | | | Number | |
|---|---|---|---|---|---|---|
| | ovulation | | Number | | of eating | |
| | damages/bud | | of eggs/bud | | damages/bud | |
| Treatment | (mean) | | (mean) | | (mean) | |
| O | 0.11 | (0.051) | 0.25 | (0.223) | 0.21 | (0.058) |
| P | 0.03 | (0.034) | 0.07 | (0.089) | 0.07 | (0.047) |
| N | 0.07 | (0.068) | 0.20 | (0.262) | 0.11 | (0.016) |

Method 2—after 12 Hours

Table 3 shows the number of beetles in rape a half day after spraying (counted 28/6)

TABLE 3

| Section | O | P | N |
|---|---|---|---|
| I | 12 | 6 | 7 |
| II | 14 | 3 | 2 |
| III | 8 | 2 | 3 |
| IV | 16 | 4 | 6 |

Table 4 shows the number of beetles in white mustard a half day after spraying (counted 28/6)

TABLE 4

| Section | O | P | N |
|---|---|---|---|
| I | 12 | 3 | 7 |
| II | 18 | 3 | 4 |
| III | 16 | 0 | 2 |
| IV | 11 | 0 | 4 |

Method 2—after 24 Hours

Table 5 shows the number of beetles in rape a whole day after spraying (counted 29/6)

TABLE 5

| Section | O | P | N |
|---|---|---|---|
| I | 20 | 6 | 10 |
| II | 17 | 7 | 14 |
| III | 11 | 0 | 10 |
| IV | 28 | 8 | 17 |

Table 6 shows the number of beetles in white mustard a whole day after spraying (counted 29/6)

TABLE 6

| Section | O | P | N |
|---|---|---|---|
| I | 29 | 6 | 17 |
| II | 26 | 10 | 9 |
| III | 18 | 8 | 9 |
| IIV | 26 | 5 | 8 |

Table 7 shows the number of beetles in rape a half day after spraying (counted 3/7)

TABLE 7

| Section | O | P | N |
|---|---|---|---|
| I | 32 | 10 | 17 |
| II | 26 | 7 | 13 |
| III | 17 | 2 | 8 |
| IV | 19 | 8 | 3 |

Table 8 shows the number of beetles in white mustard a half day after spraying (counted 3/7)

TABLE 8

| Section | O | P | N |
|---|---|---|---|
| I | 15 | 4 | 6 |
| II | 9 | 1 | 5 |
| III | 5 | 1 | 3 |
| IV | 19 | 2 | 10 |

Table 9 shows the number of beetles in rape a whole day after spraying (counted 4/7)

TABLE 9

| Section | O | P | N |
|---|---|---|---|
| I | 32 | 23 | 35 |
| II | 17 | 12 | 15 |
| III | 7 | 7 | 12 |
| IV | 20 | 8 | 11 |

Table 10 shows the number of beetles in white mustard a whole day after spraying (counted 4/7)

TABLE 10

| Section | O | P | N |
|---|---|---|---|
| I | 12 | 10 | 8 |
| II | 5 | 6 | 10 |
| III | 18 | 6 | 15 |
| IV | 43 | 23 | 28 |

### Example 5

Control of Hop Diseases, Powdery Mildew (Sphaerotheca humuli) and Downy Mildew (Pseudoperonospora humuli)

A trial site was set up for the 1999 season on a half acre of fertile sandy loam near Newent, Gloucestershire, and was managed by a professional horticulturist. The site was planted with new roots in March, Challenger (for powdery mildew) and First Gold (for downy). The hops were trained up plastic netting supported by a simple wirework trellis six feet high.

The new site gave the opportunity to run screening tests with lower disease pressures than previously experienced, but some presence of disease would be required to make the necessary assessments. A total of 11 chemical treatments were selected for each disease, and spray applications were made using a knapsack motorised mist blower at volumes of 30 to 50 gallons per acre. The disease assessments were thorough, requiring detailed examination of at least 20 leaves (typically the 6th, i.e. young leaf, for all except the basal leaves in the downy trial), or 200 hop cones per plot. Each plot was a seven-metre panel of ‘hedge’, and there were two plots for each treatment. The spray treatments, disease assessments and data handling were performed by Oxford Agricultural Trials Ltd. (registered with MAFF PSD as an official research company).

Powdery mildew had to be introduced to the site on pots of young Goldings, and the Challenger became infected by early June. Leaf disease was not rampant, but the developing hop cones were rapidly infected where they were left unprotected. The downy mildew, however, was already present at high levels within the rootstocks and was epidemic across the site by this time.

The results of these two trials have given useful comparisons i.e. experimental results from one season at one site. It is important to note that the treatments were tested by the NHA (National Hop Association of England) on behalf of growers. As can be seen, No. 10 i.e. a composition according to the present invention (“Puricide”), made a very good performance.

Results for Both Powdery and Downy Screens are Presented as Follows:

1. Treatment list of chemicals tested (table 11).

2. Summary of disease control by each treatment (table 12).

3. Summary progress report for each chemical (table 13).

National Hop Association of England Chemical Screening—1999

NHA CHEMICAL SCREENING FOR
POWDERY MILDEW CONTROL -1999

| | | Product Rate | | rate of |
|---|---|---|---|---|
| Treatment | Product | per hectolitre | | active ingredi- |
| number | Name | (100 litres) | | ent(s) in product |
| 1 | Amistar | 100 | mls | azoxystrobin (25%) |
| 2 | Product B | 75 | g | confidential to Novartis |
| 3 | Systhane 20 | 30 | mls | myclobutanil (20%) |
| 4 | Systhane 20 + | 30 | mls | myclobutanil (20%) + |
| | half rate Torch | 50 | mls | spiroxamine (50%) |
| 5 | half rate Fortress + | 8 | mls | quinoxyfen (50%) + |
| | half rate Torch | 50 | mls | spiroxamine (50%) |
| 6 | Sulphur Flowable | 600 | ml | sulphur (50%) |
| 7 | double rate Sulphur | 1200 | mls | sulphur (50%) |
| 8 | Sulphur Flowable + | 600 | ml + | sulphur (50%) + |
| | Cuprokylt | 300 | g | copper |
| | | | | oxychloride (50%) |
| 9 | FertigroSil | 2000 | ml | potassium metasilicate |
| | | | | fertiliser |
| 10 | Puritas | 3333 | ml | puricide organic fertiliser |
| 11 | Hortichem | 1000 | ml | petroleum oil (71%) |
| | Spraying Oil | | | |
| 12 | untreated | x | | x |

National Hop Association of England Chemical Screening—1999

| | | Control of Leaf | Control of Leaf | Protection of Hop |
|---|---|---|---|---|
| Trt. | Powdery | Infection before | Infection after start | Cones from disease |
| No. | Treatments - 1999 | burr | of burr | infection |
| 1 | Amistar | Fair | Fair | Fair |
| 2 | Product B | Good | Good | Good |
| 3 | Systhane 20 | Fair | Very Good | Fair |
| 4 | Systhane 20 + | Good | Good | Very Good |
| | half rate Torch | | | |
| 5 | half rate Fortress + | Fair | Very Good | Very Good |
| | half rate Torch | | | |
| 6 | Sulphur | Poor | Poor | Very Poor |
| 7 | double rate Sulphur | Good | Very Good | Fair to Good |
| 8 | Sulphur + Copper | Good | Fair | Good |
| 9 | FertigroSil | Fair | Fair | Good to Fair |
| 10 | Puricide | Good | Good | Good |
| 11 | Spraying Oil | Fair | Fair | Poor |
| 12 | Challenger- | Powdery mildew was | Rapid infection of new | Infection caused |
| | untreated | introduced to the new | leaf during July, with | deformed cones |
| | | plantation on 6 pots of | no slowing of disease | throughout their |
| | | infected Goldings in | spread during dry spell | development. Certain |
| | | May. Disease first | of weather. Infection | treatments appeared to |
| | | appeared in untreated | preferred to colonise | remove natural plant |
| | | Challenger on 1st | burr, which have no | waxes, laying the hops |
| | | June, but did not | protective wax as | more open to disease |
| | | accelerate until the | natural defense. | infection. |
| | | start of burr. | | |

National Hop Association of England Chemical Screening—1999

| | Powdery | | |
|---|---|---|---|
| Trt. | Treatments- | | |
| No. | 1999 | Summary Control in 1999 trial | Summary Progress Report - January 2000 |
| 1 | Amistar | Confirmed again as showing some limited | Noted as weak against powdery mildew in German |
| | | eradication of powdery mildew, but slow to start and | trials. In the unlikely event of receiving approval for |
| | | too weak to consider for further trials. | UK hops, its use to control downy control will also |
| | | | give some secondary control of powdery. |
| 2 | Product B | Against expectations, its purely protective action | Registration being sought in both USA and |
| | | showed promise with lower disease pressure. | Germany. Although it performed poorly against |
| | | Noticeable lasting protection of cones through | downy mildew (in a high infection trial), its appears |
| | | September; 4 weeks after the last spray treatment. | useful against powdery mildew provided disease |
| | | Future trials need to investigate leaf scorch. | levels are not high. |
| 3 | Systhane 20 | After a slow start its activity appeared to favour the | Off label approval granted in 1999, replacing |
| | | hot dry spell in July, after which control started to | Systhane 6 Flo. German trials note that it performs |
| | | slip in the wet August, and cone protection was | better in the early parts of the season, |
| | | poor, falling off sharply in September. | but is poorly at protecting the cones. |
| 4 | Systhane 20 + | The short-lived eradicant action of the half rate | Spiroxamine is noted for strong eradicant action and |
| | half rate Torch | spiroxamine gave much needed improvement to | being short-lived. Being pursued for registration on |
| | | Systhane; although cone protection had fallen off by | hops world-wide. UK will have to rely on mutual |
| | | late September. | recognition of German approval; |
| | | | trials are underway. |
| 5 | half rate | Although the level of control was not ‘perfect’, this | All the necessary trials have started in Germany and |
| | Fortress + | was simply the best of the treatments tested for both | USA in urgent prusuit of this the most effective |
| | half rate Torch | leaf and cone protection; even at half-rates of both | chemical against mould yet to be tested. Again, UK |
| | | chemicals applied on a 14 day spray interval. | approval will have to rely on mutual recognition; |
| | | | earliest use in UK 2003. |
| 6 | Sulphur | Little use against mould on its own, at this rate, and | Only permitted before cone development - no use |
| | | on a 14 day interval (a 7 day interval would be more | afterwards, certainly at this rate and with long spray |
| | | appropriate). Clearly no useful protection of cones. | intervalsl |
| | | Sprays perhaps enough to remove protective leaf | |
| | | wax without leaving enough Sulphur to prevent re- | |
| | | invasion. | |
| 7 | double rate | Huge improvement in performance by doubling the | Perhaps not commercially acceptable to use quite |
| | Sulphur | rate. Leaf protection was good by virtue of being | such a high rate. Partial success in demonstrating |
| | | harsh and scorch caused early leaf drop in August, | that higher rate can substitute shorter spray interval. |
| | | which was not commercially acceptable. Cone | Need to learn more about using this |
| | | protection encouraging. | ‘organic’ natural chemical. |
| 8 | Sulphur + Copper | More a standard practice mixture for Sulphur under | Standard practice ‘organic’ treatment is likely to be |
| | | low disease pressure. Generally good; except when | much more successful on a 7 rather than a 14 day |
| | | disease pressure high in July, and cones were given | interval; so can be most useful provided that the |
| | | no lasting protection beyond the first week of | variety has some natural ‘horizontal’ resistance to |
| | | September. | powdery mildew and sources of innoculum are |
| | | | minimised. |
| 9 | FertigroSil | First treatment to be infected after the treatment, but | A horticultural fertiliser salt given a good test and |
| | | disease was reduced by eradicant action of the | ranking just below the sulphur plus copper ‘organic’ |
| | | potassium salt. Repeated sprays caused premature | treatment. Future success likely to be from lower |
| | | leaf drop in August of older leaves - needs testing | rates with shorter spray intervals. |
| | | further. | |
| 10 | Puricide | A most encouraging new agent being developed for | Quite clearly this new Swedish ‘organic’ treatment |
| | | the organic market. Bulky rates required, and further | requires further more detailed testing to confirm |
| | | plant safety tests are needed as some leaf scorch | these encouraging results. |
| | | measured. Very surprising cone protection well into | |
| | | September. | |
| 11 | Spraying Oil | Some eradication of leaf infection, but very short- | More likely to be of use earlier in the season, when |
| | | lived. Initial cone protection fell off very rapidly. | it demostrated some useful eradicant properties. |
| | | Surprisingly no significant leaf scorch measured. | Needs testing in more detail to establish best use |
| | | Needs more detailed testing in future trials. | pattern. |
| 12 | Challenger- | Clear differences between the treatments noted | Control of mould is so much more successful where |
| | untreated | throughout the season, with a relatively small | disease levels are very low (preferably absent!). |
| | | amount of disease compared to trials in previous | There are no imminent registrations for new |
| | | years. Note how leaf disease fell off in August as | chemicals on hops, so attention must be turned to |
| | | leaves hardened and burr was preferentially infected. | removing all sources of infection. This has to be |
| | | The most important measurements were cone | accompanied by prophylactic protection measures |
| | | protection to 8th September, but comparing the | because once infection is seen, it has already been |
| | | longevity of some treatments is illuminating. | spreading for a few days. |

### Example 6

Fatty Acid Content of a Composition According to the Present Invention

The fatty acid content of a composition according to the present invention was assessed and is given below in table 4

TABLE 4
Fatty acid content in % of composition

| | | | Puricide | | |
|---|---|---|---|---|---|
| | | | % non- | | 000222a |
| | | | sat- | Sign: | % > = |
| LID No. E1639-00 | | Compound: | urated | % di- and tri | tetra non- |
| Fatty acid name | | % saturated | (mono) | non-saturated | saturated |
| Caproic acid | C6 | | | | |
| Caprylic acid | C5 | | | | |
| Capric acid | C10 | | | | |
| Lauric acid | C12 | | | | |
| Myristic acid | C14 | 0.1 | | | |
| Myristoleic acid | C14:1 | | | | |
| Pentadecane acid | C15 | | | | |
| | C15:1 | | | | |
| Palmitic acid | C16 | 10.6 | | | |
| Palmitoleic acid | C16:1 | | 0.1 | | |
| Margaric acid | C17 | 0.1 | | | |
| Heptadecene acid | C17:1 | | 0.1 | | |
| Stearic acid | C18 | 4.3 | | | |
| Oleic acid | C18:1 | | 24.3 | | |
| Linoleic acid | C18:2 | | | 51.0 | |
| Alpha-linoleic acid | C18:3 | | | 6.8 | |
| gamma-linoleic acid | C18:3 | | | | |
| Oktadekatetraene | C18:4 | | | | |
| Arachic acid | C20 | 0.4 | | | |
| Gadoleic acid | C20:1 | | 0.3 | | |
| Eicosadieneic acid | C20:2 | | | | |
| | C20:3 | | | | |
| | C20:4 | | | | |
| | C20:5 | | | | |
| Beheic acid | C22 | 0.5 | | | |
| | C22:1 | | 0.2 | | |
| | C22:4 | | | | |
| | C22:5 | | | | |
| | C22:6 | | | | |
| Lignoseric acid | C24 | 0.1 | | | |
| Tetracosene acid | C24:1 | | | | |
| Total | | 16.1% | 25.0% | 57.8% | |
| | | | | Total | 98.9% |

The fatty acids comprised in the composition are normally saponificated; preferably at least 98% of said fatty acids are normally saponificated. The fatty acids may be comprised in a vegetable oil, preferably a soybean oil. Preferably the composition has an amount of approximately 2% (weight) C₈ (caprylic acid), 2% (weight) C₁₀ (capric acid) and 2% (weight) C₁₄ (myristic acid), wherein the content of C18 is lowered with 6%. Preferably the composition has an amount of approximately 6% (weight) with fatty acids ranging (when regarding the number of carbons) from C₆ (caproic acid) to C₁₄ (myristic acid), wherein the content of C18 is lowered with 6%.

It should be understood that modifications may be made to the embodiments disclosed herein. Therefore the above description should not be construed as limiting, but merely as exemplification of preferred embodiments. Those skilled in the art will envision other modifications within the scope of the claims appended hereto.

1. An antibacterial, non-toxic and bio-degradable composition, comprising as an active ingredient at least one saponificated vegetable oil or one or more components thereof; and a carrier, wherein said carrier is water and said at least one saponificated vegetable oil or component thereof is a fatty acid containing from 6 to 18 carbons.

2. A composition according to claim 1, wherein the vegetable oil is selected from the group consisting of soybean oil, rape-seed oil and sunflower oil or a mixture thereof.

3. A composition according to claim 1 or 2, wherein the composition has an altered fatty acid pattern in comparison to the fatty acid pattern normally associated with the vegetable oil in question.

4. A composition according to claim 3 wherein the amount of said at least one saponificated vegetable oil is from 0.01 to 50% (weight).

5. A composition according to claim 3 wherein the amount of said at least one saponificated vegetable oil is from 0.1 to 5% (weight).

6. A composition according to claim 3 wherein the amount of said at least one saponificated vegetable oil is from 2 to 4% (weight).

7. A composition according to any one of claims 1 to 6 comprising an amount of approximately 2% (weight) C₈, 2% (weight) C₁₀, 2% (weight) C₁₂ and/or 2% (weight) C₁₄.

8. A composition according to any one of claims 1 to 6 comprising an amount of approximately 6% (weight) with fatty acids ranging (when regarding the number of carbons) from C₆ to C₁₄.

9. A composition according to claim 1, wherein the composition contains an increased amount of at least two fatty acids having short carbon chains, preferably one fatty acid with a carbon chain having 12 carbons and one fatty acid with a carbon chain having 14 to 18 carbons.

10. A composition according to claim 1, wherein the saponificated vegetable oil is potassium oleate.

11. A composition according to claim 1 comprising:
a) 0.1 to 50% (weight) of a saponificated vegetable oil, preferably potassium oleate,
b) optionally one or more other fatty acid(s) which may be saponificated, and
c) water, to a total of 100% (weight);
preferably 25% (weight) potassium oleate and 75% (weight) water.

12. A composition according to any one of the preceding claims, further characterized by that it also is effective for and suitable for use as a pesticide, for repelling pests and/or inhibiting the ovulation of pests.

13. A ready-to-use composition comprising a composition according to any one of claims 1 to 11, wherein the composition is diluted with water in a ratio of 1:20 to 1:40.

14. Process for the production of a composition according to claim 1, comprising saponification of a vegetable oil, preferably soybean oil, by adding an alkali hydroxide thereto, preferably potassium hydroxide; and adding a carrier.

15. Process according to claim 14, wherein the carrier is water.

16. Use of a saponificated vegetable oil, preferably saponificated soybean oil, or one or more components thereof for the manufacture of a medicament.

17. Use of a saponificated vegetable oil, preferably soybean oil, or one or more components thereof for the manufacture of a medicament for the treatment, prevention or management of bacterial, viral or fungal diseases, preferably bacterial diseases.

18. Use of a saponificated vegetable oil, preferably soybean oil, or one or more components thereof for inhibiting the ovulation of pests or for repelling of pests.

19. Method for controlling bacteria on pests comprising the application of an antibacterially effective amount of a composition according to any one of claims 1 to 10 onto said pests.

20. Method according to claim 19 wherein the pests are arthropod, nematode, helminth and protozoan pests, scabies, insects, thrips, mites, weevils, aphids, pear suckers, ants, termites, flies, ticks, dandruff, pollen (blossom) beetle, lice or fleas, preferably flies, lice, ticks, pollen beetle or fleas.

21. Method according to claim 19 wherein said pests are infesting or suspected to infest an animal or a human being, or are infesting or suspected to infest a locus, preferably a stable or a barn.

22. Method according to claim 19 wherein said pests are fungi, preferably powdery mildew, downy mildew or scurf.

23. Method according to claim 19 wherein said pests are infesting or suspected to infest an animal, a human being or crops, or are infesting or suspected to infest a locus, preferably a stable, a barn or a field for cultivation.
